(19) United States
(12) Patent Application Publication
(10) Pub. No.: US 2007/0122439 A1
(43) Pub. Date: May 31, 2007

(54) Test medium, system and method for the evaluation of surfaces

(76) Inventor: Jutta Schafer, Russelsheim (DE)

Correspondence Address: GENERAL MOTORS CORPORATION;LEGAL STAFF, MAIL CODE 482-C23-B21, P O BOX 300, DETROIT, MI, 48265-3000, US

(21) Appl. No.: 11/430,520

(22) Filed: May 9, 2006

(30) Foreign Application Priority Data
Nov. 25, 2005 (DE) 20 2005 018 444.6

(51) Int. Cl. G01N 33/44; A01N 25/00

(52) U.S. Cl. 424/405; 436/85

(57) ABSTRACT

The present invention provides a test medium for the evaluation of the resistance of surfaces comprising at least about 20 % by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide. Furthermore, the present invention describes a test specimen, a test system and a test method for the evaluation of the resistance of surfaces.

### FIELD OF THE INVENTION

The present invention relates to test media for the evaluation of the resistance of surfaces. Moreover, the present invention relates to a test specimen, whose surface is wetted with the test medium and test systems for the evaluation of the resistance of surfaces.

### BACKGROUND OF THE INVENTION

The interior materials of vehicles are exposed to various loads. Particularly, these parts are exposed to very diverse chemicals which can lead to a change of the optical characteristics of the exposed surface. It is, for example, well known that many components of insect repellents, cleaning agents, sun creams, etc., can attack the surface of interior parts.

In the state of the art, different attempts are described in order to obtain an evaluation about the resistance of the interior materials to these chemicals.

Thus, the GM specification GMN 10033 describes a test system using a certain commercial available sun cream as test medium. Unfavourably, the composition of this commercial sun cream is very complex. In the case of a change of the composition of this specified product, different problems may occur. These problems concern the reproducibility and the comparability of older test results. For example, the older product could be reproduced, in order to solve these problems. However, this is very complex and expensive, whereby the production of such product is difficult in small quantities. Furthermore, if the specified sun cream is changed, a new specification could be developed. However, in this case the comparability with older test results is not ensured.

Moreover, the GM specification GMN 10033 provides only evaluations about the resistance of the interior materials in relation to certain kinds of compositions, i.e., sun creams. However, since different compositions, for example, insect repellents, can comprise other components, different tests would have to be accomplished in each case, in order to receive a complete impression of the behavior of interior materials during load.

However, a test solution should not have too high, unrealizable demands against the resistance of a surface. The interior materials must meet a multiplicity of requirements including the costs of the materials. If a test solution is too aggressive in comparison with the compositions with which the interior materials come generally into contact, only very few probably expensive materials pass the test. Everyday life suited lower-priced materials, however, not meeting the too high requirements of the test medium would not be considered.

Consequently, it is an object of the present invention to provide a test medium which permits a reliable evaluation of the resistance of the surfaces of interior materials in relation to different compositions, with which these interior materials are contacted in the everyday life. Moreover, the test medium should be produced in a simple and economical manner. Furthermore, it is an object of the present invention to provide a test specimen, whose surface is wetted with the test medium. Additionally, it is an object of the present invention to provide a test system for the evaluation of the resistance of surfaces. Moreover, it is an object of the present invention to provide a method for the evaluation of the resistance of surfaces. Various other objects and advantages of this invention will be apparent upon reading the disclosure which follows hereinafter.

### SUMMARY OF THE INVENTION

These objects are achieved by the teaching of claims 1, 10, 15 and 16, respectively. Preferred embodiments are subject matter of the dependent claims.

The test medium for the evaluation of the resistance of surfaces comprises at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide.

The test specimen comprises a surface being wetted with a test medium for the evaluation of the resistance of surfaces comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide.

The test system for the evaluation of the resistance of surfaces comprises a device for heating and at least a test specimen comprising a surface being wetted with a test medium for the evaluation of the resistance of surfaces comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide.

A method for the evaluation of the resistance of surfaces comprises the step of applying a test medium comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide onto the surface of a test specimen.

### DETAILED DESCRIPTION OF THE INVENTION

The compound 3-(4-methoxyphenyl)-2-propenoic acid-2-ethylhexyl ester is also known under the designation octyl methoxycinnamate. The compound 3-(4-methoxyphenyl)-2-propenoic acid-2-ethylhexyl ester possesses the CAS No. 5466-77-3 and has the structural formula (I)

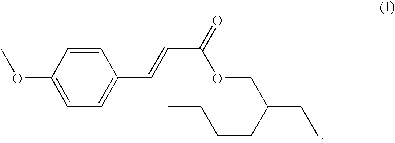

Preferably, the test medium comprises 20 to 40% by weight, more preferably 20 to 30% by weight 3-(4-methoxyphenyl)-2-propenoic acid-2-ethylhexyl ester.

The compound 2-ethylhexyl-2-cyano-3,3-diphenylacrylate is also called octocrylene. The compound 2-ethylhexyl-2-cyano-3,3-diphenylacrylate possesses the CAS No. 6197-30-4 and has the structural formula (II)

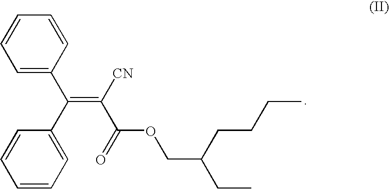

According to a preferred embodiment of the present invention, the test medium may comprise 20 to 40% by weight, more preferably 20 to 30% by weight 2-ethylhexyl-2-cyano-3,3-diphenylacrylate.

3,3,5-trimethylcyclohexyl salicylate is also known under the designation homosalate. 3,3,5-trimethylcyclohexyl salicylate possesses the CAS No. 118-56-9 and has the structural formula (III).

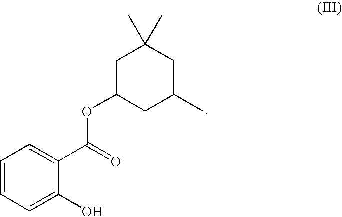

The test medium may preferably comprise 20 to 40% by weight, more preferably 20 to 30% by weight 3,3,5-trimethylcyclohexyl salicylate.

The compound N,N-diethyl-m-toluamide (DEET) possesses the CAS no. 134-62-3 and has the structural formula (IV).

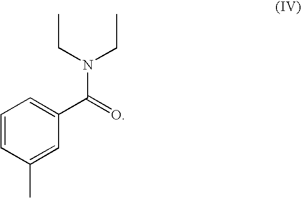

Preferably, the test medium comprises 20 to 40% by weight, more preferably 20 to 30% by weight N,N-diethyl-m-toluamide.

Preferably, the weight ratio of 3-(4-methoxyphenyl)-2-propenoic acid-2-ethylhexyl ester to 2-ethylhexyl-2-cyano-3,3-diphenylacrylate is in the range of 1.2:1 to 0.8:1, more preferably in the range of 1.1:1 to 0.9:1.

Preferably, the weight ratio of 3-(4-methoxyphenyl)-2-propenoic acid-2-ethylhexyl ester to 3,3,5-trimethylcyclohexyl salicylate is in the range of 1.2:1 to 0.8:1, more preferably in the range of 1.1:1 to 0.9:1.

Preferably, the weight ratio of 3-(4-methoxyphenyl)-2-propenoic acid-2-ethylhexyl ester to N,N-diethyl-m-toluamide is in the range of 1.2:1 to 0.8:1, more preferably in the range of 1.1:1 to 0.9:1.

Preferably, the weight ratio of 2-ethylhexyl-2-cyano-3,3-diphenylacrylate to 3,3,5-trimethylcyclohexyl salicylate is in the range of 1.2:1 to 0.8:1, more preferably in the range of 1.1:1 to 0.9:1.

Preferably, the weight ratio of 2-ethylhexyl-2-cyano-3,3-diphenylacrylate to N,N-diethyl-m-toluamide is in the range of 1.2:1 to 0.8:1, more preferably in the range of 1.1:1 to 0.9:1.

Preferably, the weight ratio of 3,3,5-trimethylcyclohexyl salicylate to N,N-diethyl-m-toluamide is in the range of 1.2:1 to 0.8:1, more preferably in the range of 1.1:1 to 0.9:1.

The test medium of the present invention is in particular suited for the investigation of the resistance of the surface of test specimens against chemical effect of compositions. Therefore, a test specimen comprising a surface being at least partly wetted with a test medium as mentioned above is a subject matter of the present invention. The test specimens can be produced in particular by using the same materials, which are used also for the production of interior parts, e.g., interior parts of vehicles. Particularly, these materials include woven fabrics or non-woven fabrics, which can be made of natural and/or synthetic fibers, leather materials and articles and/or foils, which comprise thermoplastic polymers, as for example polyolefins, e.g., polyethylene (PE) and polypropylene (PP), polycarbonates (PC), polyamides (PA), polyurethanes (PU), polyesters, e.g., polyethylene terephthalate (PET) and polybutylene terephthalate (PBT), acrylonitrile butadiene styrene copolymers (ABS) and thermoplastic elastomers, e.g., thermoplastic elastomers on basis of polyether ester (TE(PEEST)) or thermoplastic elastomers on basis of polyolefins (TEO), for example thermoplastic elastomers on basis of ethyl propylene copolymers (TPO). These polymers can be used individually or as a mixture of two or more, the blends may consist of the polymers specified above or the blends may comprise further polymers not being mentioned above.

In accordance with preferred embodiments of the present invention, the test specimens may comprise coatings, in particular with regard to the materials for interiors of vehicles. Preferred coatings are lacquers, for example, lacquers on water basis or solvent lacquers.

Furthermore, a test system comprising at least a test specimen described above and a device for heating is a subject of the present invention. Devices for heating being useful for the purpose of the present invention are well known. This device serves for even and constant heating of test specimens in order to provide preferred test conditions. Preferably, the heating device should heat up the sample test specimen to at least 60° C., more preferably at least 80° C., whereby this temperature should be kept constant ±3° C. For example a commercial available warming cupboard, drying oven or laboratory oven can be used as device for heating.

Additionally, the present invention provides a method for the evaluation of the resistance of surfaces, comprising the step of applying a test medium comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide onto the surface of a test specimen.

Preferably, a heating step is applied to the test specimen having a surface wetted with the test medium. Preferably, the test specimen is heated to a temperature of at least about 70° C., more preferably to a temperature in the range of 75° C. to 95° C. Preferably, the test specimen is heated for at least about 20 minutes, more preferably for a period of time in the range of 30 minutes to 90 minutes.

The amount of test medium applied on the test specimen is not critical. Consequently, the whole test specimen can be immersed into the test medium. However, with regard to economical considerations usually a smaller amount is sufficient for performing the method of the present invention. On the other hand, the amount should be sufficient to impart a visible change on the surface of the test specimen. Preferably, at least 20 μl of the test medium is applied on the surface of the test specimen. More preferably, the amount of test medium being applied on the surface of the test specimen is in the range of 30 μl to 100 μl.

For example, the resistance of materials, e.g., interior materials for vehicles, against the effect of chemicals, which can lead to a change of the optical characteristics of the loaded surface, can be examined by applying a small quantity of the test medium, for example about 50 μl, on the surface of a corresponding test specimen.

Subsequently, the test specimen wetted with the test medium as mentioned above is heated on 80° C±3° C. for approximately one hour. After removing the test medium, the resistance of the test specimen can be determined by visual evaluation.

The test specimen can be cleaned by methods well known is the art in order to facilitate the evaluation of the results. Preferably, the evaluation of the resistance of surfaces can be achieved visually by using an appropriate classification comprising, for example, at least two, three, four or five levels which are linked with a specified variation in the appearance of the test medium. For example, such classification can comprise the levels 0 to 5 as mentioned in Table 1.

TABLE 1

| Level | Change |
|---|---|
| 0 | No change |
| 1 | Hardly visible change, for example, a weak mark |
| 2 | Visible change of the surface, for example, a clear |
| | mark, but no swelling |
| 3 | Clearly visible change of the surface, for example, a |
| | small swelling of the surface |
| 4 | Strongly visible change of the surface, for example, a |
| | strong swelling of the surface, a small formation of |
| | wrinkles |
| 5 | Very strong visible change of the surface, for example, |
| | a strong formation of wrinkles, a separation of the |
| | lacquer |

The test medium of the present invention provides a surprisingly good evaluation to the stability of interior materials against a multiplicity of different compositions with which the interior materials can be contacted in the everyday life. These compositions include, for example, insect repellants, cleaning agents, sun creams.

The present invention will be further understood by reference to the following Examples which are given to illustrate this invention but not in any way to limit its scope.

### EXAMPLE 1

A test specimen made of thermoplastic polymers (ABS/PC; ®Bayblend T-85) comprising a lacquer of the company Schramm Coatings GmbH (Hydrodekorlack no. 05-3669) was wetted with 50 μl of a test medium of the present invention consisting of 25% by weight 3-(4-methoxyphenyl)-2-propenoic acid-2-ethylhexyl ester, 25% by weight 2-ethylhexyl-2-cyano-3,3-diphenylacrylate, 25% by weight 3,3,5-trimethylcyclohexyl salicylate and 25% by weight N,N-diethyl-m-toluamide.

Subsequently, the wetted test specimen was heated for one hour to 80° C. After this heating, the test specimen was cooled on ambient temperature and cleaned.

Thereafter, the appearance of the test specimen was examined visually, whereby an evaluation of the change was made on the basis of the levels as stated in Table 1.

In accordance with this classification the test specimen of Example 1 achieved the level 2.

### COMPARATIVE EXAMPLE 1

Essentially, Example 1 was repeated. However, a test medium consisting of N,N-diethyl-m-toluamide has been used. The test specimen of Comparative Example 1 achieved the level 5 of the classification according to Table 1.

### COMPARATIVE EXAMPLE 2

Essentially, Example 1 was repeated. However, a test medium consisting of 2-ethylhexyl-2-cyano-3,3-diphenylacrylate. The test specimen of Comparative Example 2 achieved the level 0 of the classification according to Table 1.

### COMPARATIVE EXAMPLE 3

Essentially, Example 1 was repeated. However, a test medium consisting of 3,3,5-trimethylcyclohexyl salicylate. The test specimen of Comparative Example 3 achieved the level 0 of the classification according to Table 1.

### COMPARATIVE EXAMPLE 4

Essentially, Example 1 was repeated. However, a test medium consisting of 0.25 g 3-(4-methoxyphenyl)-2-propenoic acid-2-ethylhexyl ester, 0.25 g 2-ethylhexyl-2-cyano-3,3-diphenylacrylate, 0.25 g 3,3,5-trimethylcyclohexyl salicylate, 0.25 g N,N-diethyl-m-toluamide and 3 g of a basis cream (“Cremor basalis DAC”). The test specimen of Comparative Example 4 achieved the level 1 of the classification according to Table 1.

### REFERENCE EXAMPLE 1

Essentially, Example 1 was repeated. However, the test has been performed by using Coppertone Kids (a commercially available sun cream) as test medium. The test specimen of Reference Example 1 achieved the level 2 of the classification according to Table 1.

### REFERENCE EXAMPLE 2

Essentially, Example 1 was repeated. However, the test has been performed by using a commercially-available insect repellent (Autan Family Zeckenschutz) as test medium. The test specimen of Reference Example 2 achieved the level 3 of the classification according to Table 1.

### REFERENCE EXAMPLE 3

Essentially, Example 1 was repeated. However, the test has been performed by using Coppertone Waterbabies (a commercially-available sun cream) as test medium. The test specimen of Reference Example 3 achieved the level 3 of the classification according to Table 1.

### EXAMPLE 2

Essentially, Example 1 was repeated. However, the test was performed by using a test specimen being coated with another lacquer of the company Schramm Coatings GmbH (Hydrosoftlack no. 05-3675). The test specimen of Example 2 achieved the level 4 of the classification according to Table 1.

### COMPARATIVE EXAMPLE 5

Essentially, Example 2 was repeated. However, a test medium consisting of N,N-diethyl-m-toluamide has been used. The test specimen of Comparative Example 5 achieved the level 5 of the classification according to Table 1.

### COMPARATIVE EXAMPLE 6

Essentially, Example 2 was repeated. However, a test medium consisting of 2-ethylhexyl-2-cyano-3,3-diphenylacrylate. The test specimen of Comparative Example 6 achieved the level 1 of the classification according to Table 1.

### COMPARATIVE EXAMPLE 7

Essentially, Example 2 was repeated. However, a test medium consisting of 3,3,5-trimethylcyclohexyl salicylate. The test specimen of Comparative Example 7 achieved the level 1 of the classification according to Table 1.

### COMPARATIVE EXAMPLE 8

Essentially, Example 2 was repeated. However, a test medium consisting of 0.25 g 3-(4-methoxyphenyl)-2-propenoic acid-2-ethylhexyl ester, 0.25 g 2-ethylhexyl-2-cyano-3,3-diphenylacrylate, 0.25 g 3,3,5-trimethylcyclohexyl salicylate, 0.25 g N,N-diethyl-m-toluamide and 3 g of a basis cream (“Cremor basalis DAC”). The test specimen of Comparative Example 8 achieved the level 1 of the classification according to Table 1.

### REFERENCE EXAMPLE 4

Essentially, Example 2 was repeated. However, the test has been performed by using Coppertone Kids (a commercially-available sun cream) as test medium. The test specimen of Reference Example 4 achieved the level 4 of the classification according to Table 1.

### REFERENCE EXAMPLE 5

Essentially, Example 2 was repeated. However, the test has been performed by using a commercially-available insect repellent (Autan Family Zeckenschutz) as test medium. The test specimen of Reference Example 5 achieved the level 4 of the classification according to Table 1.

### REFERENCE EXAMPLE 6

Essentially, Example 2 was repeated. However, the test has been performed by using Coppertone Waterbabies (a commercially-available sun cream) as test medium. The test specimen of Reference Example 6 achieved the level 4 of the classification according to Table 1.

In consideration of commercial-available sun creams and insect repellents, the examples, comparative examples and reference examples clearly show that the test medium of the present invention provides an improved evaluation of the resistance of the surface of a test specimen in comparison to the use of the single compounds or mixtures on the basis of other conventional components of sun creams.

The test specimen of Example 1 provides very similar results as the reference examples. Comparative Example I shows a too high sensitivity, while Comparative Examples 3 and 4 show a too low sensitivity.

1. A test medium for the evaluation of the resistance of surfaces comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide.

2. The test medium of claim 1, wherein the test medium comprises 20 to 40% by weight 3-(4-methoxyphenyl)-2-propenoic acid-2-ethylhexyl ester, 20 to 40% by weight 2-ethylhexyl-2-cyano-3,3-diphenylacrylate, 20 to 40% by weight 3,3,5-3,3,5-trimethylcyclohexyl salicylate and 20 to 40% by weight N,N-diethyl-m-toluamide.

3. The test medium of claim 2, wherein the test medium consists of 20 to 30% by weight 3-(4-methoxyphenyl)-2-propenoic acid-2-ethylhexyl ester, 20 to 30% by weight 2-ethylhexyl-2-cyano-3,3-diphenylacrylate, 20 to 30% by weight 3,3,5-3,3,5-trimethylcyclohexyl salicylate and 20 to 30% by weight N,N-diethyl-m-toluamide.

4. The test medium of claim 1, wherein the weight ratio of 3-(4-methoxyphenyl)-2-propenoic acid-2-ethylhexyl ester to 2-ethylhexyl-2-cyano-3,3-diphenylacrylate is in the range of 1.2:1 to 0.8:1.

5. The test medium of claim 1, wherein the weight ratio of 3-(4-methoxyphenyl)-2-propenoic acid-2-ethylhexyl ester to 3,3,5-trimethylcyclohexyl salicylate is in the range of 1.2:1 to 0.8:1.

6. The test medium of claim 1, wherein the weight ratio of 3-(4-methoxyphenyl)-2-propenoic acid-2-ethylhexyl ester to N,N-diethyl-m-toluamide is in the range of 1.2:1 to 0.8:1.

7. The test medium of claim 1, wherein the weight ratio of 2-ethylhexyl-2-cyano-3,3-diphenylacrylate to 3,3,5-trimethylcyclohexyl salicylate is in the range of 1.2:1 to 0.8:1.

8. The test medium of claim 1, wherein the weight ratio of 2-ethylhexyl-2-cyano-3,3-diphenylacrylate to N,N-diethyl-m-toluamide is in the range of 1.2:1 to 0.8:1.

9. The test medium of claim 1, wherein the weight ratio of 3,3,5-trimethylcyclohexyl salicylate to N,N-diethyl-m-toluamide is in the range of 1.2:1 to 0.8:1.

10. A test specimen comprising a surface being wetted with a test medium for the evaluation of the resistance of surfaces comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least 5 about 20% by weight 3,3,5-trimethylcyclohexyl salicylate and at least about 20% by weight N,N-diethyl-m-toluamide.

11. The test specimen of claim 10, wherein the test specimen comprises thermoplastic resins.

12. The test specimen of claim 11, wherein the test specimen comprises polypropylene, polycarbonate and/or acrylnitrile butadiene styrene copolymers.

13. The test specimen of claim 10, wherein the test specimen comprises a coating.

14. The test specimen of claim 13, wherein the coating is obtained by lacquering of the specimen with a lacquer on the basis of water or a solvent lacquer.

15. A test system for the evaluation of the resistance of surfaces, wherein the test system comprises a device for heating and at least a test specimen comprising a surface being wetted with a test medium for the evaluation of the resistance of surfaces comprising at least about 20% by weight 5 3-(4-methoxyphenyl)-2-propenoic acid-2-ethylhexyl ester, at least about 20% by weight 2-ethylhexyl-2-cyano-3,3-diphenylacrylate, at least about 20% by weight 3,3,5-trimethylcyclohexyl salicylate and at least about 20% by weight N,N-diethyl-m-toluamide.

16. A method for the evaluation of the resistance of surfaces, comprising the step of applying a test medium comprising at least about 20% by weight 3-(4-methoxyphenyl)-2-propenoic acid-2-ethylhexyl ester, at least about 20% by weight 2-ethylhexyl-2-cyano-3,3-diphenylacrylate, at least about 20% 5 by weight 3,3,5-trimethylcyclohexyl salicylate and at least about 20% by weight N,N-diethyl-m-toluamide onto the surface of a test specimen.

17. The method according to claim 16, wherein the method comprises the step of heating the test specimen having a surface wetted with the test medium.

18. The method according to claim 17, wherein the test specimen is heated to at least about 70° C.

19. The method according to claim 18, wherein the test specimen is heated to a temperature in the range of 75° C. to 95° C.

20. The method according to claim 17, wherein the test specimen is heated for at least 20 minutes.

21. The method according to claim 20, wherein the test specimen is heated for a period of time in the range of 30 minutes to 90 minutes.

22. The method according to claim 16, wherein at least 20 μl of the test medium is applied on the surface of the test specimen.

23. The method according to claim 22, wherein the amount of test medium being applied on the surface of the test specimen is in the range of 30 μl to 100 μl.
